(19) United States
(12) Patent Application Publication
(10) Pub. No.: US 2006/0283706 A1
(43) Pub. Date: Dec. 21, 2006

(54) Biosensor arrangement and method for producing it

(75) Inventors: Bela Kelety, Frankfurt am Main (DE); Wolfgang Dorner, Hochheim (DE); Robin Krause, Frankfurt am Main (DE)

Correspondence Address: D. PETER HOCHBERG CO. L.P.A., 1940 EAST 6TH STREET, CLEVELAND, OH, 44114, US

(73) Assignee: IonGate Biosciences GmbH, Frankfurt am Main (DE)

(21) Appl. No.: 11/454,494

(22) Filed: Jun. 16, 2006

(30) Foreign Application Priority Data
Jun. 17, 2005 (DE) 10 2005 028 245.8

(51) Int. Cl. G01N 33/487

(52) U.S. Cl. 204/403.01

(57) ABSTRACT

A biosensor arrangement and a method for producing a biosensor arrangement. The biosensor arrangement comprises at least one electrode substrate with a surface which forms an electrode of the biosensor arrangement. At least one biomaterial area is formed which is provided in and/or on the surface of the electrode substrate. The electrode substrate is formed with or of a synthetic material or with or of a polymer material, which in turn is electrically conductive.

### RELATED APPLICATION

This application claims foreign priority based on German Application Serial No. 10 2005 028 245.8, filed on Jun. 17, 2005, the content of which is incorporated herein by reference in its entirety.

### BACKGROUND OF THE INVENTION

1. Field of the Invention

This invention relates to a biosensor arrangement and a method for producing it.

2. Description of the Prior Art

In many areas of the chemical and biochemical analysis technology, biocompatible or biologically compatible material arrangements are used, in particular biocompatible or biologically compatible sensor arrangements or sensor electrode arrangements. Through these material arrangements, sensor arrangements or sensor electrode arrangements, certain measuring processes are performed in the application in terms of a chemically, biologically or biochemically relevant analyte.

In analysis processes with high throughputs, e.g. with so-called high throughput screening processes, different characteristics are desirable for the biologically compatible material arrangements, sensor arrangements or sensor electrode arrangements, in particular in terms of their electrical sensitivity, their mechanical stability and/or their high and economical availability. With customary material arrangements, sensor arrangements or sensor electrode arrangements, carrier substrates are used which are, actually, comparatively mechanically stable in design, but otherwise entail comparatively difficult handling and which also are not necessarily economically produced.

### SUMMARY OF THE INVENTION

This invention is based on the objective of providing a biosensor arrangement as well as a method for producing it which are especially easy, economical and yet reliable to handle.

This problem is solved by the biosensor arrangement according to the present invention. Furthermore, the problem is solved by a method for producing a biosensor arrangement according to the invention. Advantageous developments are respectively the subject of the claims and discussed herein.

The biosensor arrangement according to the invention is designed for the amperometric and/or potentiometric, pharmacological testing of the site of action and/or the agent. The biosensor arrangement according to the invention comprises at least one electrode substrate with a surface which forms an electrode of the biosensor arrangement or a part of an electrode. Furthermore, a biomaterial area is formed which is provided in and/or on the surface of the electrode substrate. The electrode substrate is formed with or of a synthetic material or with or of a polymer material. According to the invention, the synthetic material or the polymer material is electrically conductive.

It is thus a core idea of this invention to use as electrode substrate or as a part thereof a synthetic material or a polymer material which is, in turn, electrically conductive. Through this measure, the customarily provided metallic structures or structures on the basis of conductive metal oxides can be left for standard electrode substrates. This has process-technical, metrological and/or possibly biochemical advantages since, in many cases, it cannot be excluded that the usually provided metal electrodes emit metallic traces into the environment and thus lead to a contamination of the biological objects to be examined by the biosensor arrangement. This is prevented in accordance with the invention.

A biomaterial area as defined by the invention should be understood above and in the following as a material area which is compatible with a species to be examined and thus to be added to the biosensor arrangement according to the invention, said species being in particular in the form of eukaryotic cells, prokaryotic cells, bacteria, viruses, components thereof, membrane fragments thereof, structures thereof, each in native form, in modified form, in purified, microbiologically changed form and/or in a molecular-biologically changed form, as well as in the form of vesicles, liposomes, micellar structures and/or their components or structures and furthermore biological units contained therein, in particular in the form of membrane proteins, ion pumps, ion channels, transporters, receptors, components thereof and structures thereof.

In a development of the biosensor arrangement according to the invention, the synthetic material or the polymer material is formed with or of one or several organic materials.

In another development of the biosensor arrangement according to the invention, the electrical conductivity of the synthetic material or the polymer material is additionally or alternatively formed by providing at least one first aggregate in the synthetic material or polymer material.

In an additional development of the biosensor arrangement according to the invention, a metallic material is additionally or alternatively provided as at least one first aggregate.

With an advantageous embodiment of the biosensor arrangement according to the invention, it is additionally or alternatively provided that—as at least one first aggregate—a form of carbon is provided.

With a preferred embodiment of the biosensor arrangement according to the invention, it is additionally or alternatively provided that—as at least one first aggregate—a material of or with nanoparticles and/or with nanotubes is provided.

With another advantageous embodiment of the biosensor arrangement according to the invention, it is additionally or alternatively provided that—as at least one first aggregate—a material or a combination of materials is provided from the group which consists of carbon in the form of soot, carbon in the form of graphite, carbon in the form of nanoparticles, carbon in the form of buckminsterfullerenes, or their especially caged derivatives, carbon in the form of nanotubes and derivatives of these materials.

With a particularly preferred embodiment of the biosensor arrangement according to the invention, it is additionally or alternatively provided that the biomaterial area is formed electrically insulating and that—through the biomaterial area—the electrode substrate is formed electrically insulated.

With another preferred embodiment of the biosensor arrangement according to the invention, it is additionally or alternatively provided that the biomaterial area is formed layer-like.

With a particularly advantageous embodiment of the biosensor arrangement according to the invention, it is additionally or alternatively provided that the biomaterial area is formed with or of a succession of mono-layers.

With another embodiment of the biosensor arrangement according to the invention, it is additionally or alternatively provided that the mono-layers are formed as spontaneously self-organizing layers.

With an additional embodiment of the biosensor arrangement according to the invention, it is additionally or alternatively provided that the biomaterial area or one or several layers of the biomaterial area are formed as a chemically and/or physically modified or converted area of the surface of the electrode substrate.

With an advantageous embodiment of the biosensor arrangement according to the invention, it is additionally or alternatively provided that the biomaterial area or one or several layers of the biomaterial area are formed via a second aggregate in the material of the electrode substrate.

With another advantageous embodiment of the biosensor arrangement according to the invention, it is additionally or alternatively provided that the biomaterial area or one or several layers of the biomaterial area are formed via an inherent surface structure in the material of the electrode substrate.

With a preferred embodiment of the biosensor arrangement according to the invention, it is additionally or alternatively provided that the biomaterial area or one or several layers of the biomaterial area are formed via an additional surface material applied on the surface of the electrode substrate.

With a particularly advantageous embodiment of the biosensor arrangement according to the invention, it is additionally or alternatively provided that the biomaterial area is formed as a layer or with one top layer facing away from the electrode substrate, with or of an amphiphilic organic compound or a lipid.

Conceivable is also an alternative or additional form of embodiment of the biosensor arrangement according to the invention in which the electrode substrate and the biomaterial area are formed as a membrane biosensor electrode and functioning as a secondary carrier of the biosensor arrangement, with a plurality of primary carriers being provided in the immediate spatial vicinity of the secondary carrier and with the primary carriers comprising biological units activatable to an electrical action.

Furthermore, an alternative or additional form of embodiment of the biosensor arrangement according to the invention is also possible in which—as a primary carrier—a primary carrier is provided from the group which is formed of: eukaryotic cells, prokaryotic cells, bacteria, viruses, components thereof, membrane fragments thereof, structures thereof, each in native form, in modified form, in purified microbiologically changed form and/or in a molecular-biologically changed form.

Alternatively conceivable is also a form of embodiment of the biosensor arrangement according to the invention in which—as a primary carrier—a primary carrier is provided from the group which is formed of: vesicles, liposomes and micellar structures.

With another design of the biosensor arrangement according to the invention, it is additionally or alternatively provided that, through the biomaterial area, the pertinent electrode is, in operation, electrically insulated from a provided measuring medium, from the primary carriers and from the biological units.

With a particularly advantageous design of the biosensor arrangement according to the invention, it is additionally or alternatively provided that the area of the biomaterial area insulating and covering the electrode substrate is formed with a membrane structure or SSM with a surface of approx. A≈0.1-50 mm² and with a specific electrical conductivity of approx. Gₘ≈1-100 nS/cm² and/or with a specific capacity of approx. Cₘ≈10-1000 nF/cm².

With another form of the biosensor arrangement according to the invention, it is additionally or alternatively provided that a biological unit is provided which is formed to be activatable to an electrogenic charge carrier movement, in particular, to an electrogenic charge carrier transport.

With another preferred form of the biosensor arrangement according to the invention, it is additionally or alternatively provided that—as a biological unit—one unit each is provided from the group which is formed of: membrane proteins, ion pumps, ion channels, transporters, receptors, components thereof and structures thereof.

As a biological unit, a unit can be provided in native form, in a modified form, in a purified form, in a microbiologically changed form, or in a molecular-biologically changed form.

Furthermore, it is possible that a carrier substrate is formed with a surface, in particular on a provided top side, and that the electrode substrate is formed at least partially on and/or in the carrier substrate and/or as a part of the surface of the carrier substrate.

It is also conceivable that the carrier substrate together with the electrode substrate is formed as a vessel—in particular, in the form of a flow-through vessel, in a closed form except for, at the most, inlet and outlet, or as a part of a vessel.

The carrier substrate and the electrode substrate can be formed monolithically.

The carrier substrate and the electrode substrate can here be formed by being produced in a multi-component injection molding technique.

The carrier substrate can be formed of or with a chemically and biologically inert material.

The carrier substrate can be formed of or with an electrically insulating material.

The carrier substrate can be formed of or with a mechanically flexible material, e.g. in the type of a film.

The carrier substrate can be formed of or with an at best low-adsorptive material versus proteins, biological and/or chemical agents.

The carrier substrate can be formed of or with a material or a combination of materials from the group which consists of PMMA, PTFE, POM, FR4, polyimide, PI, kaptone, PEN, PET, materials transparent in the UV range and materials transparent in the visible spectral range.

In an advantageous manner, a plurality of electrode substrates and biomaterial areas can be formed, in a contiguous or in a separated form, electrically insulated from each other and laterally at a distance from each other, and a plurality of electrically independent electrodes can thus be formed.

The plurality of electrodes can here be arranged in a row or in matrix form.

According to another aspect of this invention, a method is developed for producing a biosensor arrangement for the amperometric and/or potentiometric, pharmacological testing of the site of action and/or the agent, in which the biosensor arrangement is formed with at least one electrode substrate with a surface, the electrode substrate forming an electrode of the biosensor arrangement or a part of an electrode. Furthermore, at least one biomaterial area is formed which is provided in and/or on the surface of the electrode substrate. The electrode substrate is formed with or of a synthetic material or with or of a polymer material, the synthetic material or the polymer material being electrically conductive.

In a development of the method according to the invention for producing the biosensor arrangement according to the invention, the synthetic material or the polymer material is formed with or of one or several organic materials.

In another development of the method according to the invention for producing the biosensor arrangement according to the invention, the electrical conductivity of the synthetic material or the polymer material is additionally or alternatively formed by providing at least one first aggregate in the synthetic material or the polymer material.

In an additional development of the method according to the invention for producing the biosensor arrangement according to the invention, a metallic material is additionally or alternatively provided as at least one first aggregate.

With an advantageous embodiment of the method according to the invention for producing the biosensor arrangement according to the invention, it is additionally or alternatively provided that—as at least one first aggregate—a form of carbon is provided.

With a preferred embodiment of the method according to the invention for producing the biosensor arrangement according to the invention, it is additionally or alternatively provided that—as at least one first aggregate—a material of or with nanoparticles and/or with nanotubes is provided.

With another advantageous embodiment of the method according to the invention for producing the biosensor arrangement according to the invention, it is additionally or alternatively provided that—as at least one first aggregate—a material or a combination of materials is provided from the group which consists of carbon in the form of soot carbon in the form of graphite, carbon in the form of nanoparticles, carbon in the form of buckminsterfullerenes, or their especially caged derivatives, carbon in the form of nanotubes and derivatives of these materials.

With a particularly preferred embodiment of the method according to the invention for producing the biosensor arrangement according to the invention, it is additionally or alternatively provided that the biomaterial area is formed electrically insulating and that—through the biomaterial area—the electrode substrate is formed electrically insulating.

With another preferred embodiment of the biosensor arrangement according to the invention, it is additionally or alternatively provided that the biomaterial area is formed layer-like.

With a particularly advantageous embodiment of the method according to the invention for producing the biosensor arrangement according to the invention, it is additionally or alternatively provided that the biomaterial area is formed with or of a succession of mono-layers.

With another embodiment of the method according to the invention for producing the biosensor arrangement according to the invention, it is additionally or alternatively provided that the mono-layers are formed as spontaneously self-organizing layers.

With an additional embodiment of the method according to the invention for producing the biosensor arrangement according to the invention, it is additionally or alternatively provided that the biomaterial area or one or several layers of the biomaterial area are formed as a chemically and/or physically modified or converted area of the surface of the electrode substrate.

With an advantageous embodiment of the method according to the invention for producing the biosensor arrangement according to the invention, it is additionally or alternatively provided that the biomaterial area or one or several layers of the biomaterial area are formed via a second aggregate in the material of the electrode substrate.

With another advantageous embodiment of the method according to the invention for producing the biosensor arrangement according to the invention, it is additionally or alternatively provided that the biomaterial area or one or several layers of the biomaterial area are formed via an inherent surface structure in the material of the electrode substrate.

With a preferred embodiment of the method according to the invention for producing the biosensor arrangement according to the invention, it is additionally or alternatively provided that the biomaterial area or one or several layers of the biomaterial area are formed via an additional surface material applied on the surface of the electrode substrate.

With a particularly advantageous embodiment of the method according to the invention for producing the biosensor arrangement according to the invention, it is additionally or alternatively provided that the biomaterial area is formed as a layer or with one top layer facing away from the electrode substrate, with or of an amphiphilic organic compound or a lipid.

Conceivable is also an alternative or additional form of embodiment of the method according to the invention for producing the biosensor arrangement according to the invention in which the electrode substrate and the biomaterial area are formed as a membrane biosensor electrode and functioning as a secondary carrier of the biosensor arrangement, with a plurality of primary carriers being provided in the immediate spatial vicinity of the secondary carrier and with the primary carriers comprising biological units activatable to an electrical action.

Furthermore, an alternative or additional form of embodiment of the method according to the invention for producing the biosensor arrangement according to the invention is also possible in which—as a primary carrier—a primary carrier is provided from the group which is formed of: eukaryotic cells, prokaryotic cells, bacteria, viruses, components thereof, membrane fragments thereof, structures thereof, each in native form, in modified form, in purified, microbiologically changed form and/or in a molecular-biologically changed form.

Alternatively conceivable is also a form of embodiment of the method according to the invention for producing the biosensor arrangement according to the invention in which—as a primary carrier—a primary carrier is provided from the group which is formed of: vesicles, liposomes and micellar structures.

With another embodiment of the method according to the invention for producing the biosensor arrangement according to the invention, it is additionally or alternatively provided that, through the biomaterial area, the pertinent electrode is, in operation, electrically insulated from a provided measuring medium, from the primary, carriers and from the biological units.

With a particularly advantageous design of the method according to the invention for producing the biosensor arrangement according to the invention, it is additionally or alternatively provided that the area of the biomaterial area insulating and covering the electrode substrate is formed with a membrane structure or SSM with a surface of approx. A≈0.1-50 mm² and with a specific electrical conductivity of approx. Gₘ≈1-100 nS/cm² and/or with a specific capacity of approx. Cₘ≈10-1000 nF/cm².

With another form of the method according to the invention for producing the biosensor arrangement according to the invention, it is additionally or alternatively provided that a biological unit is provided which is formed to be activatable to an electrogenic charge carrier movement, in particular, to an electrogenic charge carrier transport.

With another preferred form of the biosensor arrangement according to the invention, it is additionally or alternatively provided that—as a biological unit—one unit each is provided from the group which is formed of: membrane proteins, ion pumps, ion channels, transporters, receptors, components thereof and structures thereof.

As a biological unit, a unit can be provided in native form, in a modified form, in a purified form, in a microbiologically changed form, or in a molecular-biologically changed form.

Furthermore, it is possible that a carrier substrate is formed with a surface, in particular on a provided top side, and that the electrode substrate is formed at least partially on and/or in the carrier substrate and/or as a part of the surface of the carrier substrate.

It is also conceivable that the carrier substrate together with the electrode substrate is formed as a vessel—in particular, in the form of a flow-through vessel, in a closed form except for, at the most, inlet and outlet, or as a part of a vessel.

The carrier substrate and the electrode substrate can be formed monolithically.

The carrier substrate and the electrode substrate can here be formed by being produced in a multi-component injection molding technique.

The carrier substrate can be formed of or with a chemically and biologically inert material.

The carrier substrate can be formed of or with an electrically insulating material.

The carrier substrate can be formed of or with a mechanically flexible material, e.g. in the type of a film.

The carrier substrate can be formed of or with an at best low-material adsorptive material versus proteins, biological and/or chemical agents.

The carrier substrate can be formed of or with a material or a combination of materials from the group which consists of PMMA, PTFE, POM, FR4, polyimide, PI, kaptone, PEN, PET, materials transparent in the UV range and materials transparent in the visible spectral range.

In an advantageous manner, a plurality of electrode substrates and biomaterial areas can be formed, in a contiguous or in a separated form, electrically insulated from each other and laterally at a distance from each other, and a plurality of electrically independent electrodes can thus be formed.

The plurality of electrodes can here be arranged in a row or in matrix form.

These and other aspects of this invention also result, in other words, from the following statements.

The invention is used, inter alia, for the simplification and cost reduction in sensor manufacture.

A measuring room is, e.g. a material impermeable to water from its surrounding and/or impermeable to solvents, the room having a defined, fillable volume and a surface suitable for establishing a hybrid biosensor, the surface comprising a material which is electrically conductive and contacted toward the outside with regard to the interior measuring room.

Measuring rooms are known whose walls are formed, for example, of a round glass tube section, glued onto a planar piece of glass and of the planar piece of glass. Concentrically arranged with the glass tube section, a thin-layer technically produced gold layer is provided on the piece of glass, in the form of a round column of, for example, 3 mm in diameter and 200 nm in height. This gold layer is connected, for example, via a bridge of gold—with a width of 200 μm and a height of 200 nm which extends under the glued-on glass tube section—with a circular ring of gold arranged concentrically to the glass tube, outside of the glass tube section.

Moreover, measuring rooms are known whose bottoms consist of printed circuit board material. Gold-plated circuit board conductors of copper are used as the basis for establishing a surface for attaching protein-containing lipid membrane structures. The other walls of the measuring rooms are formed of plastic or polymer tube pieces or glass tube sections.

Moreover, measuring rooms are known which are monolithically produced of plastic or polymer materials, either by machining or by injection molding. As a basis for building up a surface for attaching protein-containing lipid membrane structures, gold-plated metal pins are used which are glued into the work pieces.

Solid supported biomimetic structures are known which are produced by the self-structuring application of amphiphilic molecules on hydrophilic substrate surfaces of conductive materials (example for substrate materials: indium tin oxide, ITO for short, glass carbon). With the exception of lipids from extremophilic organisms, amphiphilic molecules generally form—depending on the type of production of the said materials—a monomolecular layer which is hydrophobic on its surface facing away from the substrate surface, or a molecular double layer which is hydrophilic on its surface facing away from the substrate surface.

The described materials ITO and glass carbon, as well as gold or gold-plated metal parts comprises hydrophilic surfaces, as a rule. This has two consequences. Firstly, in the combination with polymers suitable for mass production, there is the risk of boundary layer effects which can result in the destabilization of cementing, to a detachment and/or a capillary effect of aqueous test solutions. Secondly, the preparation of sensor surfaces is complicated and error-prone because a hydrophilic surface must always be assumed which, as a rule, is first coated—for preparation of the use as a biosensor—with a hydrophobizing monomolecular layer and subsequently with a hydrophilizing monomolecular layer.

One aspect of the invention is the use of an electrically conductive organic polymer as a supporting solid for establishing hybrid biosensors. An electrically conductive organic polymer can here be understood—not exclusively, but especially—a thermoplastic elastomer material made conductive through the addition of carbon.

Another aspect of the invention is the modification of the polymer surface which is used for the purpose of ensuring a stable attachment of protein-containing lipid membrane structures.

Another aspect concerns the form and the composition of sensors. Polymers or plastic materials which are not electrically conductive can be used to create a measuring room as a component part of a sensor. Electrically conductive polymers or plastic materials can then be used or used at the same time to form active sensor surfaces. With sensors established in this manner, there are no contact surfaces between plastics or polymers and non-plastics or non-polymers.

Due to the inherent properties of conductive polymers which can be very similar to those of non-conductive thermoplastic elastomers, both materials can be combined with each other, especially with the multi-component injection molding technique. This mode of production renders later joining and/or gluing superfluous and prevents the boundary effects occurring when metal parts are combined with plastic materials such as, for example, the capillary effect of aqueous test solutions.

A hydrophobicity of conductive polymers allows the direct attachment of a mono-layer of amphiphilic molecules, with an outwardly hydrophilic surface developing which—depending on the selection of the amphiphilic substance—is excellently suitable for the attachment of protein-containing lipid membrane structures.

The addition of further additives can be used to develop a suitable surface for the direct attachment of protein-containing lipid membrane structures after the conclusion of the production process.

A number of sensors was prepared by gluing granulate of conductive polymers into the bores of a printed circuit board. A glue was used which was hardened out under UV light. The printed circuit board was glued under a 96-sample vessel micro-titer plate. After hardening of the glues, a solution of diphytanoyl-phosphatidylcholine in decane was given into the sample vessels and largely removed immediately after wetting the surface of the conductive polymer. The sample vessel was then filled with test buffer. After the almost complete removal of the test buffer, the vessel was filled with a protein-containing membrane suspension or, respectively, a liposome suspension and incubated for several hours at 4° C. The illustrated measuring signals were obtained in solvent change experiments with the measuring solutions provided for the corresponding proteins (NhaA in liposomes or, respectively, EAAC1 in CHO cell membrane fragments).

### BRIEF DESCRIPTION OF THE DRAWINGS

In the following, this invention will be described in more detail by diagrammatic drawings on the basis of preferred exemplary embodiments.

FIG. 1A is a diagrammatic top view of a sectional side view of a first embodiment of the biosensor arrangement according to the invention.

FIG. 1B is a diagrammatic sectional side view of a first embodiment of the biosensor arrangement according to the invention.

FIG. 2A is a diagrammatic top view of a second embodiment of the biosensor arrangement according to the invention.

FIG. 2B is a sectional side view of a second embodiment of the biosensor arrangement 1 according to the invention.

FIG. 3 is a diagrammatic and sectional side view, explaining a primary carrier for a biological unit as defined by the invention.

FIG. 4 is a diagrammatic and sectional side view of the details of another embodiment of the biosensor arrangement according to the invention.

FIG. 5 is a diagrammatic and sectional side view of the details of yet another embodiment of the biosensor arrangement according to the invention.

FIG. 6 is a diagrammatic and sectional side view of the details of another embodiment of the biosensor arrangement according to the invention.

FIG. 7 is a diagram demonstrating exemplary measuring curves of a first application of an embodiment of the biosensor arrangement according to the invention.

FIG. 8 is a diagram demonstrating, in the form of exemplary measuring curves, another application of an embodiment of the biosensor arrangement according to the invention.

### DETAILED DESCRIPTION OF THE INVENTION

Hereinafter, the same reference numbers designate the same, similar, or similarly acting structures or elements. A detailed description will not be repeated in every instance of their occurrence.

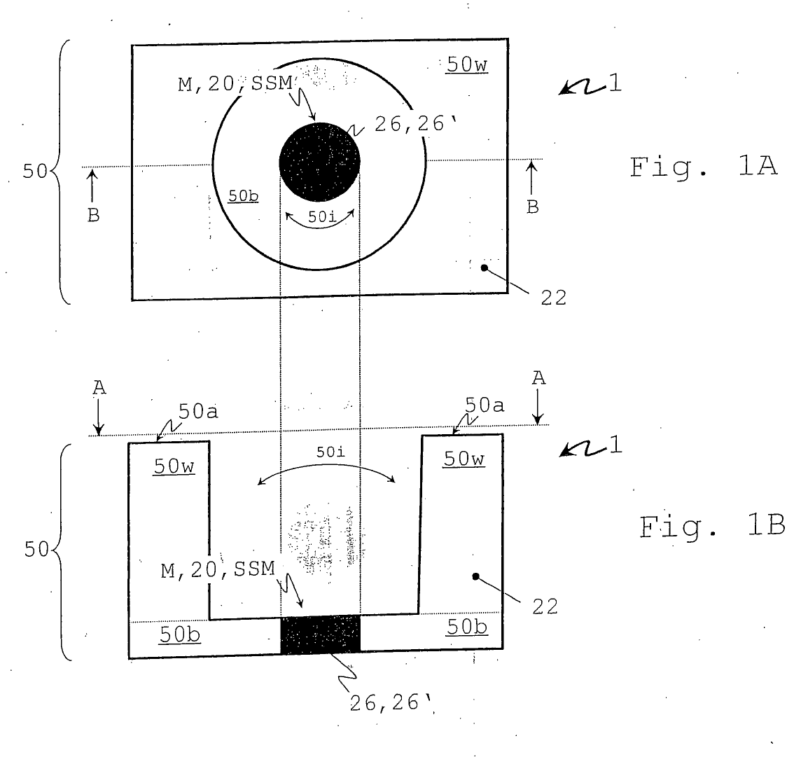

FIG. 1 shows—in a diagrammatic and sectional side view—a first embodiment of the biosensor arrangement 1 according to the invention.

FIGS. 1A and 1B show—in a diagrammatic top view or, respectively, in a diagrammatic sectional side view—a first embodiment of the biosensor arrangement 1 according to the invention.

The embodiment of the biosensor arrangement 1 according to the invention according to FIGS. 1A and 1B forms as a whole a vessel or measuring vessel 50 which can be closed; however, FIG. 1B presents the open form of the measuring vessel 50. The measuring vessel 50 is formed of wall areas or walls 50w and of a bottom area or bottom 50b. The wall areas 50w and the bottom area 50b are formed by a carrier substrate or carrier material 22. As shown in FIG. 1B, the carrier substrate 22 is formed with a recess through which the inside vessel room 50i of the vessel 50 is defined. On the free area of the bottom 50b of the vessel 50, an electrode substrate 26 is integrated into the bottom 50b for the definition of an electrode 26′. The upper side 26a of the electrode 26′ or the electrode substrate 26 faces toward the inside vessel room 50i of the vessel 50 and serves to define a membrane sensor electrode M in the form of a solid supported membrane SSM and thus a secondary carrier 20. The rear side 26b of the electrode 26′ and thus of the electrode substrate 26 can be used for external contacting and for the electrical pick-up of the electrode 26′.

FIG. 1A is a top view of the object presented in FIGS. 1A and 1B, along the plane or line A-A of FIG. 1B. Vice versa, the view of FIG. 1B results as a sectional view along the line or plane B-B of FIG. 1A.

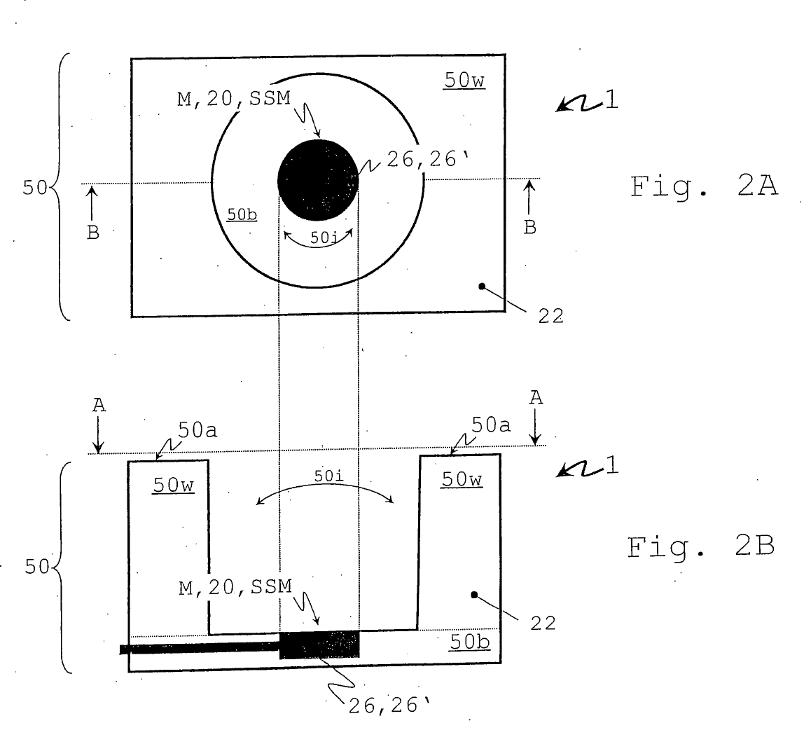

The embodiment of the biosensor arrangement 1 according to the invention which is presented in FIGS. 2A and 2B essentially corresponds with the embodiment presented in FIGS. 1A and 1B. However, there is a difference to the effect that the electrode substrate 26 only has one free surface 26a, whereas the sub-surface 26b is formed embedded in the carrier substrate 22, i.e. in the bottom area 50b, so that there is additionally a lateral electrical pickup 29 for diverting electrical signals forming on the membrane sensor electrode M in the form of SSM as a secondary carrier 20.

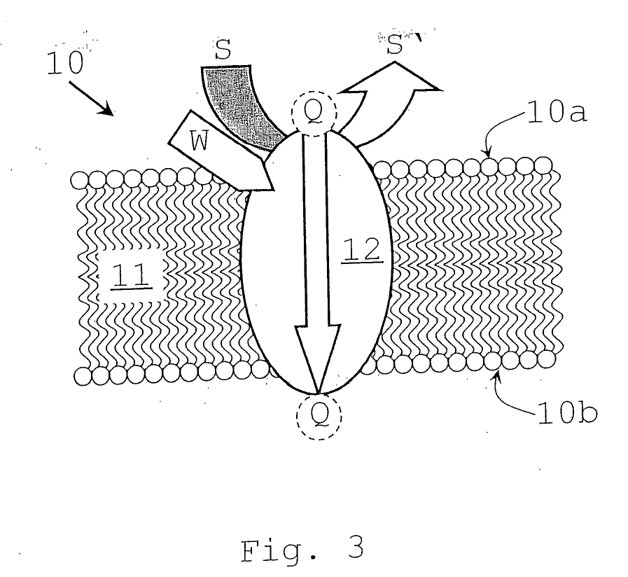

FIG. 3 explains—in a diagrammatic and sectional side view—the structure and the function of a primary carrier 10 which comprises a biological unit 12, i.e. in a membrane-spanning form, by which electrical actions can be effected.

Basic element of the primary carrier 10 is a membrane 11, according to FIG. 3 in the form of a lipid double layer. Through membrane 11, a first or upper side 1Oa will be defined, and a second or lower side lob. The sides 10a and 10b of the primary carrier 10 can be e.g. the intracellular and extracellular side of a cell membrane or organelle membrane or also of an artificial membrane. A biological unit 12 is formed membrane-spanning in the membrane 11, reaching in fact from the first side 10a to the second side 10b. As already described above, the biological unit 12 can be a membrane protein. FIG. 3 presents an embodiment in which the biological unit 12 defines an ion pump by means of which—through conversion of a first substrate species S to a converted substrate species S′—a load-carrying species Q is transported from the first side 10a in the direction of the presented transport arrow to the second side 10b of the cell membrane or membrane 11; and, if necessary, the influence of an agent W on the functionality of the biological unit 12 can be optionally used, e.g. for the purposes of examination.

As already discussed above, the primary carrier 10 may be a membrane fragment or a closed membrane arrangement within the meaning of a cell, an organelle, a virus, a bacterium, a vesicle, a liposome or a micellar structure. Conceivable are also fragments or structures of these entities.

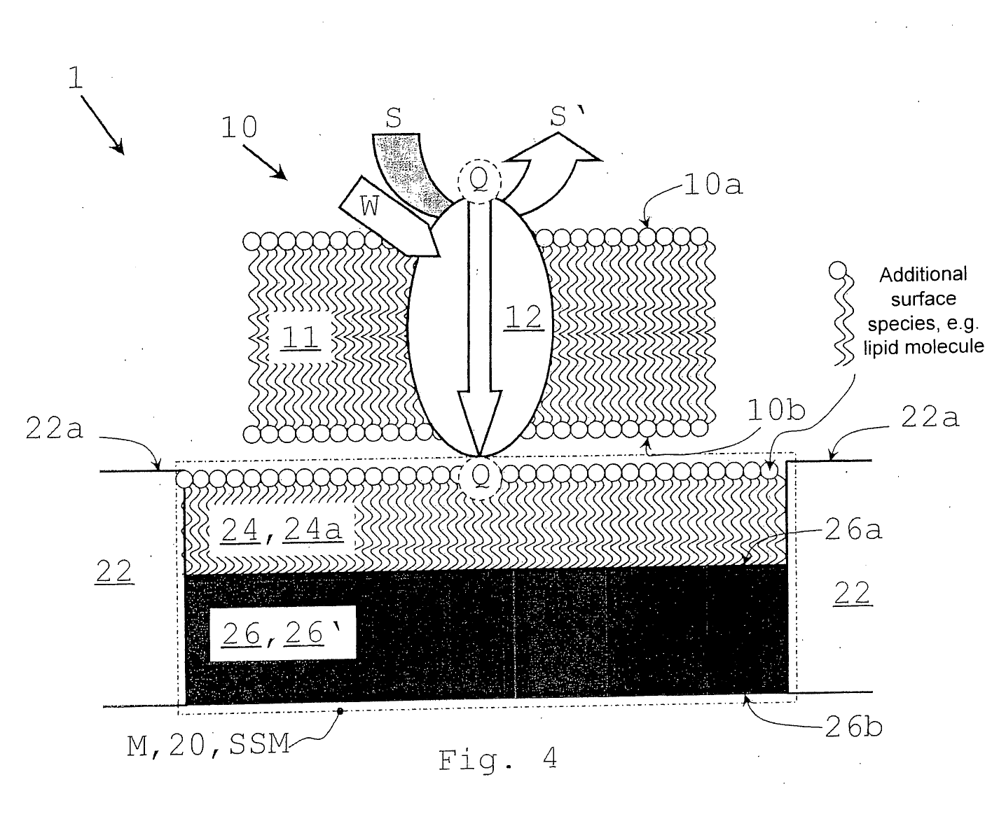

FIG. 4 shows the structure of a first application of an embodiment of the biosensor arrangement 1 according to the invention on a primary carrier 10, as it is presented in FIG. 3. The membrane biosensor electrode M in the form of a solid supported membrane SSM serves as a secondary carrier 20 for the primary carrier 10.

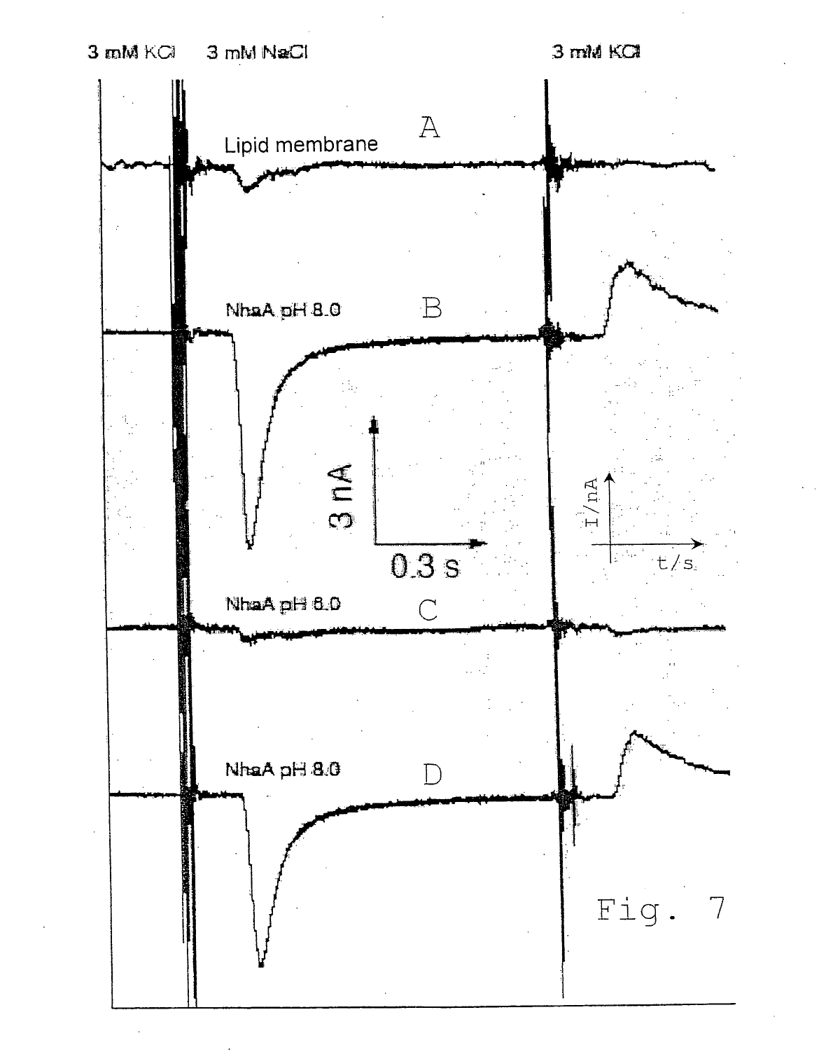

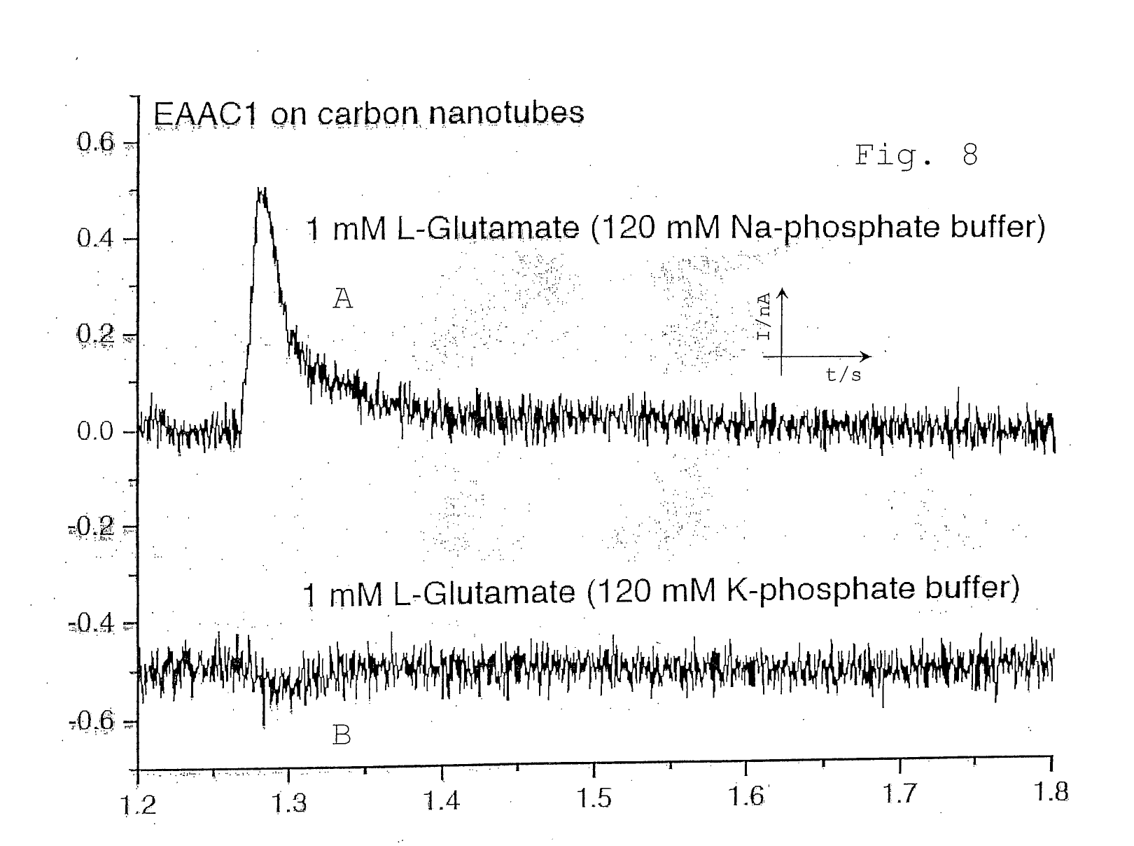

The primary carrier 10 is called primary carrier because it is the actual carrier of the biological unit 12 which is up for examination. The solid supported membrane SSM within the meaning of a membrane biosensor electrode M here serves as carrier only in a secondary sense which is why it is called secondary carrier 20. Through a spatial approximation of one or of a multitude or a plurality of primary carrier(s) 10 with at least one or a multitude or a plurality of biological unit(s) 12—in particular in an identical type and manner—a macroscopic load transport can be measured with the correspondingly identical alignment of the primary carriers 10 and the biological units 12 and with simultaneous activation, i.e. in the form of a displacement current, as will yet be explained below in connection with FIGS. 7 and 8.

This situation principally applies for all embodiments of the FIGS. 4, 5 and 6.

In the embodiment of FIG. 4, the electrode substrate 26 in the carrier substrate 22 is monolithically integrated and thus forms an electrode 26′. Versus the measuring medium 30 in which the primary carrier or carriers 10 with the biological units 12 are found, this electrode 26′ is formed electrically insulated by the biomaterial area 24—here in form of a lipid mono-layer 24a—so that the resulting arrangement of the solid supported membrane SSM as a membrane biosensor electrode M is acting as a capacitively coupled electrode. To a crucial degree this is achieved by the lipid mono-layer 24a as its sole component of the biomaterial area 24 on the hydrophobic surface 26a of the electrode substrate 26 being formed such that an electrically tight and thus electrically insulating structure exists versus the measuring medium 30, the primary carrier 10 and versus the biological unit 12.

The biomaterial area 24 should principally establish a possible compatibility between the electrode substrate 26 and the corresponding electrode 26′ and the primary carrier as well as to the biological units 12 so that—in terms of the biological function of the biological unit 12—the most natively possible conditions will be achieved.

In the embodiment of FIG. 4, the biomaterial area 24 exclusively consists of a lipid mono-layer 24a, with the lipid molecule densely packed in a two-dimensional structure on the surface 26a of the electrode substrate 26 being tightly arranged and with—moreover—the lipid chains due to their hydrophobic nature being aligned to the hydrophobic surface 26a of the electrode substrate 26, and the hydrophilic lipid heads aligned to the measuring medium 30, here in the form of an aqueous measuring medium 30.

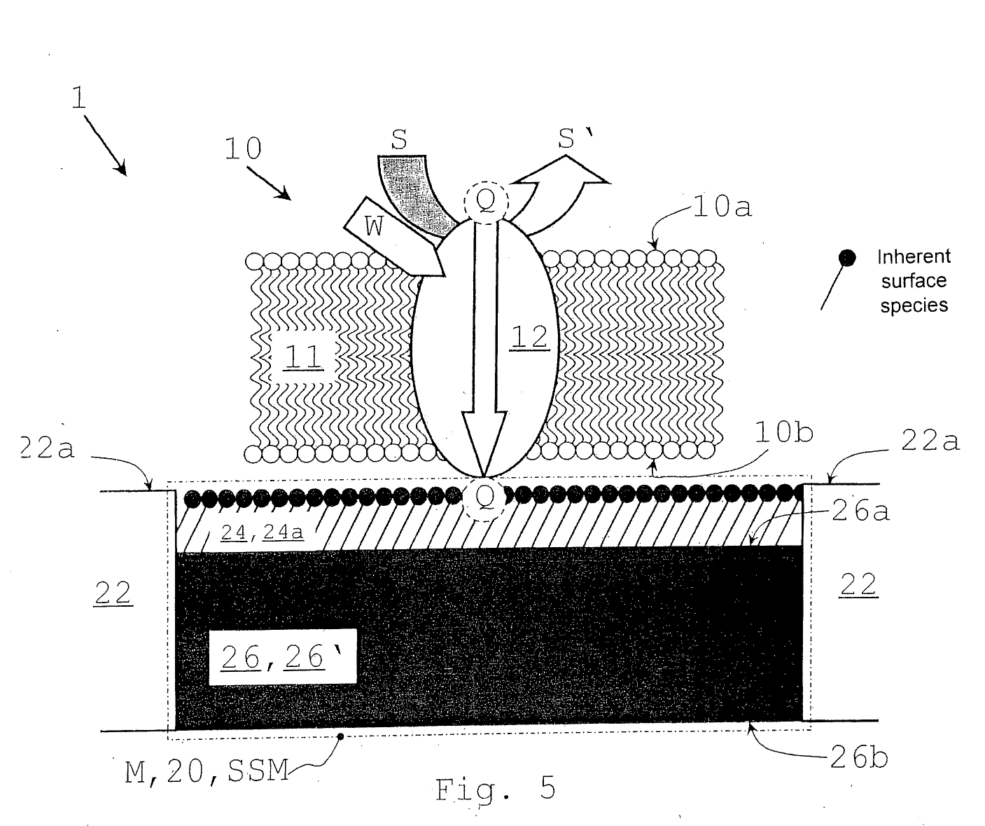

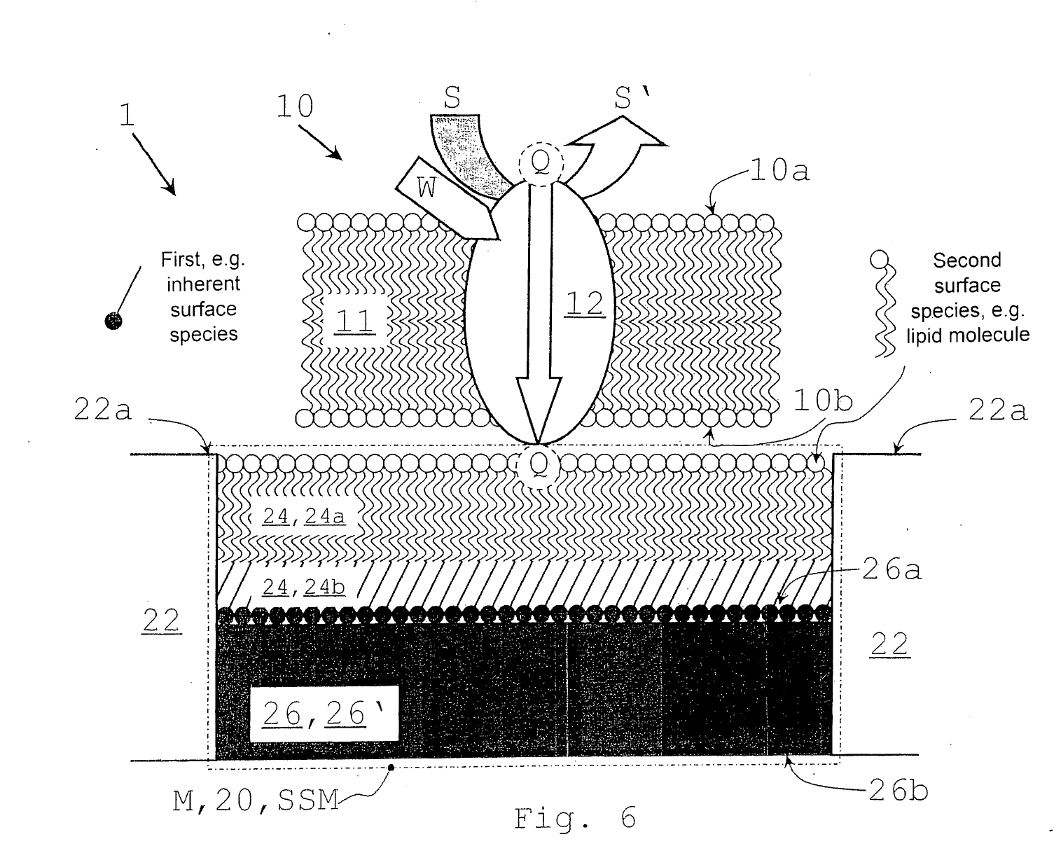

The arrangements of FIGS. 5 and 6 essentially correspond with the arrangement presented in FIG. 4, with different biomaterial areas 24 being available, however.

With the embodiment of FIG. 5, the biomaterial area 24 also consists of a single mono-layer 24a which, however—due to a second aggregate—inherently develops from the electrode substrate 26 on its surface 26a. The difference between bulk material and surface material of the electrode substrate 26 can here be particularly decisive, with the admixtures in the electrode substrate 26 in the form of aggregates unfolding their effect on the surface 26a and thus offering a biologically compatible structure for the primary carriers 10 and the biological units 12 included therein.

With the embodiment of FIG. 6, a first mono-layer 24b for the biomaterial area 24 will also be at first inherently formed directly on the surface 26a of the electrode substrate 26 via specified aggregates. The molecules of the aggregate are organizing on the surface 26a of the electrode substrate 26 such that a hydrophobic surface structure develops on which a second surface species can be arranged in the form of additionally added lipid molecules, similar to that in the embodiment of FIG. 4 so that, in turn, in the measuring medium 30, the lipid heads are arranged in the direction of the measuring medium 30 and thus, in turn, defining a biologically compatible surface of the membrane biosensor electrode M in the form of a solid supported membrane SSM as a secondary carrier 20.

FIGS. 7 and 8 show electrical currents which were generated due to biological units 12 which are arranged in primary carriers 10, e.g. within the scope of one of the arrangements from FIGS. 1 to 6. The time t in seconds s is marked off on the abscissas, and the measured electrical force of current I on the ordinates.

The measuring results presented in FIG. 7 are liposomes arranged on a biosensor arrangement 1 according to the invention, the liposomes being provided as primary carriers 10 and, as biological units 12, exhibiting a sodium proton exchanger bacterially expressed therein. Presented are in each case electrical transport currents developing through the sodium and proton transport in opposite directions on the boundary surface of the membrane biosensor electrode M as a secondary carrier 20 with a solution exchange from 3 mmol/l potassium chloride to 3 mmol/l sodium chloride. The traces A to D in FIG. 7 correspond with experiments which were achieved on a naked lipid membrane 24a, in the presence of the sodium proton exchanger at a pH value of 8.0, in the presence of the sodium proton exchanger at a pH value of 6.0 and, respectively, again in the presence of the sodium proton exchanger at a pH value of 8.0.

The experiment shown in FIG. 8 is a biosensor arrangement according to the invention in which an electrode area of an electrode substrate 26 based on an organic polymer was made conductive by means of carbon nanotubes. As measuring species, an ensemble of membrane fragments as primary carriers 10 is here provided, with the so-called EAAC1 transporter from CHO cells being provided in the membrane fragments as primary carrier 10. The experiments of traces A and B from FIG. 8 show solvent changes to a solvent without L-glutamate to 1 mmol/l glutamate, i.e. with a basic buffer of 120 mmol/l sodium sulfate in water or, respectively, 120 mmol/l potassium sulfate in water, each under corresponding buffering.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A biosensor arrangement for the amperometric and/or potentiometric, pharmacological testing of the site of action and/or the agent, said arrangement comprising:
at least one electrode substrate having a surface which forms an electrode of the biosensor arrangement or a part of an electrode, and further comprises
at least one biomaterial area provided in and/or on the surface of the electrode substrate, wherein with the electrode substrate is formed with or of material selected from the group consisting of a synthetic material and a polymer material, and wherein the synthetic material or the polymer material is electrically conductive.

2. The biosensor arrangement according to claim 1, wherein the synthetic material or the polymer material is formed with or of at least one organic material.

3. The biosensor arrangement according to claim 1, wherein the electrical conductivity of the synthetic material or the polymer material is formed by providing at least one first aggregate in the synthetic material or polymer material.

4. The biosensor arrangement according to claim 3, wherein said at least one first aggregate is a metallic material.

5. The biosensor arrangement according to claim 3, wherein said at least one first aggregate is a form of carbon.

6. The biosensor arrangement according to claim 3, wherein said at least one first aggregate is a material of or with at least one of nanoparticles and nanotubes.

7. The biosensor arrangement according to claim 3, wherein said at least one first aggregate comprises at least one material selected from the group consisting of carbon in the form of soot, carbon in the form of graphite, carbon in the form of nanoparticles, carbon in the form of buckminsterfullerenes or their especially caged derivatives, carbon in the form of nanotubes, and derivatives of these materials.

8. The biosensor arrangement according to claim 1, wherein the biomaterial area is formed electrically insulating, and the electrode substrate is formed electrically insulated, through the biomaterial area.

9. The biosensor arrangement according to claim 1, wherein the biomaterial area is formed layer-like.

10. The biosensor arrangement according to claim 1, wherein the biomaterial area is formed with or of a succession of mono-layers.

11. The biosensor arrangement according to claim 10, wherein the mono-layers are formed as spontaneously self-organizing layers.

12. The biosensor arrangement according to claim 1, wherein the biomaterial area or one or several layers of the biomaterial area are formed as at least one of a chemically and physically modified or converted area of the surface of the electrode substrate.

13. The biosensor arrangement according to claim 1, a second aggregate in the material of the electrode substrate for forming the biomaterial area or one or several layers of the biomaterial area.

14. The biosensor arrangement according to claim 1, further comprising an inherent surface structure in the material of the electrode substrate for forming the biomaterial area or one or several layers of the biomaterial area.

15. The biosensor arrangement according to claim 1, further comprising an additional surface material applied on the surface of the electrode substrate for forming the biomaterial area or one or several layers of the biomaterial area.

16. The biosensor arrangement according to claim 1, wherein the biomaterial area is formed as a layer or with one top layer facing away from the electrode substrate, with or of an amphiphilic organic compound or a lipid.

17. The biosensor arrangement according to claim 1, wherein the electrode substrate and the biomaterial area are formed as a membrane biosensor electrode and function as a secondary carrier of the biosensor arrangement, wherein a plurality of primary carriers are provided in the immediate spatial vicinity of the secondary carrier, and wherein the primary carriers comprise biological units activatable to an electrical action.

18. The biosensor arrangement according to claim 17, further comprising a primary carrier selected from the group consisting of eukaryotic cells, prokaryotic cells, bacteria, viruses, components thereof, membrane fragments thereof and structures thereof, each in a form selected from the group consisting of native form, modified form, purified form, microbiologically changed form and a molecular-biologically changed form.

19. The biosensor arrangement according to claim 17, further comprising a primary carrier selected from the group consisting of vesicles, liposomes and micellar structures.

20. The biosensor arrangement according to claim 1, wherein, through the biomaterial area, the pertinent electrode is electrically insulated from a provided measuring medium, from the primary carriers and from the biological units, while, in operation.

21. The biosensor arrangement according to claim 1, wherein the area of the biomaterial area insulating and covering the electrode substrate is formed with a membrane structure with a surface of approximately A≈0.1-50 mm² and with a specific electrical conductivity of approximately Gₘ≈1-100 nS/cm² and/or with a specific capacity of approximately Cₘ≈10-1000 nF/cm².

22. The biosensor arrangement according to claim 1, further comprising a biological unit which is activatable to an electrogenic charge carrier movement.

23. The biosensor arrangement according to claim 1, wherein as a biological unit, one unit each is provided selected from the group consisting of membrane proteins, ion pumps, ion channels, transporters, receptors, components thereof and structures thereof.

24. The biosensor arrangement according to claim 1, wherein as a biological unit, a unit can be provided in a form selected from the group consisting of a modified form, a purified form, a microbiologically changed form, and a molecular-biologically changed form.

25. The biosensor arrangement according to claim 1, further comprising a carrier substrate formed with a surface, and wherein the electrode substrate is formed at least partially on and/or in the carrier substrate and/or as a part of the surface of the carrier substrate.

26. The biosensor arrangement according to claim 25, wherein the carrier substrate together with the electrode substrate is formed as a vessel.

27. The biosensor arrangement according to claim 25, wherein the carrier substrate and the electrode substrate are formed monolithically.

28. The biosensor arrangement according to claim 27, wherein the carrier substrate and the electrode substrate are formed by being produced in a multi-component injection molding technique.

29. The biosensor arrangement according to claim 25, wherein the carrier substrate is formed of or with a chemically and biologically inert material.

30. The biosensor arrangement according to claim 25, wherein the carrier substrate is formed of or with an electrically insulating material.

31. The biosensor arrangement according to claim 25, wherein the carrier substrate is formed of or with a mechanically flexible material.

32. The biosensor arrangement according to claim 25, wherein the carrier substrate is formed of one or with an at best low-adsorptive material versus proteins, biological and/or chemical agents.

33. The biosensor arrangement according to claim 25, wherein the carrier substrate is formed of or with at least one material selected from the group consisting of PMMA, PTFE, POM, FR4, polyimide, PI, kaptone, PEN, PET, materials transparent in the UV range and materials transparent in the visible spectral range.

34. The biosensor arrangement according to claim 25, further comprising a plurality of electrode substrates and biomaterial areas in a contiguous or in a separated form, electrically insulated from each other and laterally at a distance from each other, for forming a plurality of electrically independent electrodes.

35. The biosensor arrangement according to claim 34, wherein the plurality of electrodes is arranged in a form selected from the group consisting of a row and a matrix.

36. A method for producing a biosensor arrangement for the amperometric and/or potentiometric, pharmacological testing of the site of action and/or the agent, comprising the steps of:
providing at least one electrode substrate having a surface for forming an electrode of the biosensor arrangement or apart of an electrode;
providing at least one biomaterial area in and/or on the surface of the electrode substrate; and
forming the electrode substrate with or of a material selected from the group consisting of a synthetic material and a polymer material, wherein the synthetic material or the polymer material is electrically conductive.

37. The method according to claim 36, further comprising the step of forming the synthetic material or the polymer material with or of at least one organic material.

38. The method according to claim 36, further comprising the step of forming the electrical conductivity of the synthetic material or the polymer material by providing at least one first aggregate in the synthetic material or polymer material.

39. The method according to claim 38, comprising the step of providing a metallic material as said at least one first aggregate.

40. The method according to claim 38, comprising the step of providing a form of carbon as said at least one first aggregate.

41. The method according to claim 38, comprising the step of providing a material of or with at least one of nanoparticles and nanotubes as said at least one first aggregate.

42. The method according to claim 38, comprising the step of providing at least one material selected from the group consisting of carbon in the form of soot, carbon in the form of graphite, carbon in the form of nanoparticles, carbon in the form of buckminsterfullerenes or their especially caged derivatives, carbon in the form of nanotubes, and derivatives of these materials, wherein said at least one material is provided as said at least one first aggregate.

43. The method according to claim 36, comprising the step of forming the biomaterial area as electrically insulating, and forming the electrode substrate electrically insulated, through the biomaterial area.

44. The method according to claim 36, comprising the step of forming he biomaterial area layer-like.

45. The method according to claim 36, further comprising the step of forming the biomaterial area with or of a succession of mono-layers.

46. The method according to claim 45, further comprising the step of forming said mono-layers as spontaneously self-organizing layers.

47. The method according to claim 36, further comprising the step of forming the biomaterial area or at least one layer of the biomaterial area as a chemically and/or physically modified or converted area of the surface of the electrode substrate.

48. The method according to claim 36, further comprising the step of forming the biomaterial area or at least one layer of the biomaterial area by a second aggregate in the material of the electrode substrate.

49. The method according to claim 36, further comprising the step of forming the biomaterial area or at least one layer of the biomaterial area by an inherent surface structure in the material of the electrode substrate.

50. The method according to claim 36, further comprising the step of forming the biomaterial area or at least one layer of the biomaterial area by an additional surface material applied on the surface of the electrode substrate.

51. The method according to claim 36, further comprising the step of forming the biomaterial area as a layer or with one top layer facing away from the electrode substrate, and with or of a material selected from the group consisting of an amphiphilic organic compound and a lipid.

52. The method according to claim 36, further comprising the step of forming the electrode substrate and the biomaterial area as a membrane biosensor electrode for functioning as a secondary carrier of the biosensor arrangement, and wherein said method further comprises providing a plurality of primary carriers in the immediate spatial vicinity of the secondary carrier, and forming the primary carriers with biological units activatable to an electrical action.

53. The method according to claim 52, further comprising the step of providing a primary carrier selected from the group consisting of eukaryotic cells, prokaryotic cells, bacteria, viruses, components thereof, membrane fragments thereof, structures thereof, each in a form selected from the group consisting of a native form, a modified form, a purified form, a microbiologically changed form and a molecular-biologically changed form.

54. The method according to claim 52, further comprising the step of providing said primary carrier selected from the group consisting of vesicles, liposomes and micellar structures.

55. The method according to claim 36, further comprising the step of electrically insulating, while in operation, the pertinent electrode through the biomaterial area from a provided measuring medium, from the primary carriers and from the biological units.

56. The method according to claim 36, further comprising the step of forming the area of the biomaterial area insulating and covering the electrode substrate with a membrane structure having a surface of approximately A≈0.1-50 mm² and with a specific electrical conductivity of approximately Gₘ≈1-100 nS/cm² and/or with a specific capacity of approximately Cₘ≈10-1000 nF/cm².

57. The method according to claim 36, further comprising the step of providing a biological unit activatable to an electrogenic charge carrier movement.

58. The method according to claim 36, comprising the step of providing, as a biological unit, one unit each of said biological unit selected from the group consisting of membrane proteins, ion pumps, ion channels, transporters, receptors, components thereof and structures thereof.

59. The method according to claim 36, further comprising the step of providing, as a biological unit, a unit can in a form selected from the group consisting of a native form, a modified form, a purified form, a microbiologically changed form, and a molecular-biologically changed form.

60. The method according to claim 36, further comprising the steps of forming a carrier substrate with a surface and forming the electrode substrate at least partially on and/or in the carrier substrate and/or as a part of the surface of the carrier substrate.

61. The method according to claim 60, comprising the step of forming the carrier substrate together with the electrode substrate as a vessel.

62. The method according to claim 60, comprising the step of forming the carrier substrate and the electrode substrate monolithically.

63. The method according to claim 62, further comprising the step of forming the carrier substrate and the electrode substrate by producing said carrier substrate and said electrode substrate in a multi-component injection molding technique.

64. The method according to claim 60, further comprising the step of forming the carrier substrate of or with a chemically and biologically inert material.

65. The method according to claim 60, further comprising the step of forming the carrier substrate of or with an electrically insulating material.

66. The method according to claim 60, further comprising the step of forming the carrier substrate of or with a mechanically flexible material.

67. The method according to claim 60, further comprising the step of forming the carrier substrate of one or with an at best low-adsorptive material versus proteins, biological and/or chemical agents.

68. The method according to claim 60, further comprising the step of forming the carrier substrate of or with at least one material selected from the group consisting of PMMA, PTFE, POM, FR4, polyimide, PI, kaptone, PEN, PET, materials transparent in the UV range and materials transparent in the visible spectral range.

69. The method according to claim 60, further comprising the step of forming a plurality of electrode substrates and biomaterial areas, in a contiguous or in a separated form, electrically insulated from each other and laterally at a distance from each other to form a plurality of electrically independent electrodes.

70. The method according to claim 69, further comprising the step of arranging the plurality of electrodes in a form selected from the group consisting of a row and a matrix form.

71. The biosensor arrangement according to claim 22, further comprising a biological unit which is activatable to an electrogenic charge carrier transport.

72. The biosensor arrangement according to claim 25, wherein the carrier substrate is formed on a provided top side.

73. The biosensor arrangement according to claim 26, wherein the carrier substrate together with the electrode substrate is formed in the form of a flow-through vessel, in a closed form except for—at the most—inlet and outlet, or as part of a vessel.

74. The biosensor arrangement according to claim 31, wherein the carrier substrate is formed of or with a type of a film.

75. The method according to claim 57, further comprising the step of providing a biological unit activatable to an electrogenic charge carrier transport.

76. The method according to claim 60, further comprising the step of forming a carrier substrate on a provided top side.

77. The method according to claim 61, comprising the step of forming the carrier substrate together with the electrode substrate in the form of a flow-through vessel, in a closed form except for—at the most—inlet and outlet, or as part of a vessel.

78. The method according to claim 66, further comprising the step of forming the carrier substrate of or with a type of a film.
